(19) United States
(12) Patent Application Publication
(10) Pub. No.: US 2019/0069880 A1
(43) Pub. Date: Mar. 7, 2019

(54) ULTRASOUND PROBE AND ULTRASOUND PROBE MANUFACTURING METHOD

(71) Applicant: Canon Medical Systems Corporation, Otawara-shi (JP)

(72) Inventors: Toshiyuki NAGASE, Yokohama (JP); Masaaki ISHITSUKA, Nasushiobara (JP); Toru HIRANO, Yokohama (JP)

(73) Assignee: Canon Medical Systems Corporation, Otawara-shi (JP)

(21) Appl. No.: 16/119,823

(22) Filed: Aug. 31, 2018

(30) Foreign Application Priority Data
Sep. 4, 2017 (JP) 2017-169731
Aug. 31, 2018 (JP) 2018-162576

(51) Int. Cl. A61B 8/00

(52) U.S. Cl. CPC A61B 8/4494; A61B 8/145; A61B 8/54; A61B 8/4444

(57) ABSTRACT

An ultrasound probe includes: a plurality of transducer elements each configured to perform at least one selected from between transmission of an ultrasound wave and reception of an ultrasound wave; first circuitry that is arranged on a rear face side of the transducer elements, is provided with electronic circuitry corresponding to two or more of the transducer elements organized in a first group, and is provided with a through silicon via corresponding to two or more of the transducer elements organized in a second group; and second circuitry that is arranged on a rear face side of the first circuitry, is provided with a through silicon via corresponding to the two or more of the transducer elements organized in the first group, and is provided with electronic circuitry corresponding to the two or more of the transducer elements organized in the second group.

### CROSS-REFERENCE TO RELATED APPLICATIONS

This application is based upon and claims the benefit of priority from Japanese Patent Application No. 2017-169731, filed on Sep. 4, 2017; and Japanese Patent Application No. 2018-162576, filed on Aug. 31, 2018; the entire contents of which are incorporated herein by reference.

### FIELD

Embodiments described herein relate generally to an ultrasound probe and an ultrasound probe manufacturing method.

### BACKGROUND

A known ultrasound diagnosis apparatus is configured to generate an ultrasound image that expresses an internal state of an examined subject in the image, by using, as an ultrasound probe, a two-dimensional (2D) array probe including a plurality of transducer elements and one or more large scale integration (LSI) circuitries. The large scale integration circuitries are provided with various types of electronic circuitries.

### BRIEF DESCRIPTION OF THE DRAWINGS

FIG. 1 is a diagram for explaining an exemplary configuration of an ultrasound diagnosis apparatus according to a first embodiment;

FIG. 2 is a drawing illustrating an exemplary configuration of an ultrasound probe according to the first embodiment;

FIG. 3 is a drawing for explaining an example of controlling circuitries according to the first embodiment;

FIG. 4 is a drawing for explaining an example of transmission and reception circuitry according to the first embodiment;

FIG. 5 is a drawing for explaining an example of another transmission and reception circuitry according to the first embodiment;

FIG. 6 is a drawing for explaining examples of two large scale integration circuitries according to the first embodiment;

FIG. 7 is a drawing illustrating a part of a method for manufacturing the ultrasound probe according to the first embodiment;

FIG. 8A is a drawing for explaining an example of a laminated structure of three large scale integration circuitries according to the first embodiment;

FIG. 8B is a drawing for clearly illustrating connection relationships in the example in FIG. 8A;

FIG. 9 is a drawing illustrating an example in which transmission and reception circuit and controlling circuitry are provided in each of the regions of large scale integration circuitry;

FIG. 10 is another drawing illustrating the example in which transmission and reception circuitry and controlling circuitry are provided in each of the regions of the large scale integration circuitry;

FIG. 11 is a drawing for explaining an example of an ultrasound probe according to a first modification example of the first embodiment;

FIG. 12 is a drawing illustrating an example of a part of a method for manufacturing an ultrasound probe according to a second modification example of the first embodiment;

FIG. 13A is a drawing for explaining an example of an ultrasound probe according to a second embodiment;

FIG. 13B is another drawing for explaining the example of the ultrasound probe according to the second embodiment;

FIG. 14A is a drawing for explaining an example of an ultrasound probe according to a third embodiment; and

FIG. 14B is another drawing for explaining the example of the ultrasound probe according to the third embodiment.

FIG. 14C is a drawing for explaining an another example of an ultrasound probe according to a third embodiment; and

FIG. 14B is another drawing for explaining the another example of the ultrasound probe according to the third embodiment.

### DETAILED DESCRIPTION

An ultrasound probe according to an embodiment includes a plurality of transducer elements, first circuitry, and second circuitry. The plurality of transducer elements are each configured to perform at least one selected from between transmission of an ultrasound wave and reception of an ultrasound wave. The first is arranged on a rear face side of the plurality of transducer elements. The first circuitry is provided with electronic circuitry corresponding to two or more of the plurality of transducer elements organized in a first group and is provided with a through silicon via corresponding to two or more of the plurality of transducer elements organized in a second group. The second circuitry is arranged on a rear face side of the first circuitry. The second circuitry is provided with a through silicon via corresponding to the two or more of the transducer elements organized in the first group and is provided with electronic circuitry corresponding to the two or more of the transducer elements organized in the second group.

Exemplary embodiments and modification examples of an ultrasound probe and an ultrasound probe manufacturing method will be explained in detail below, with reference to the accompanying drawings. It is possible to combine together any of the embodiments and the modification examples, as appropriate.

### First Embodiment

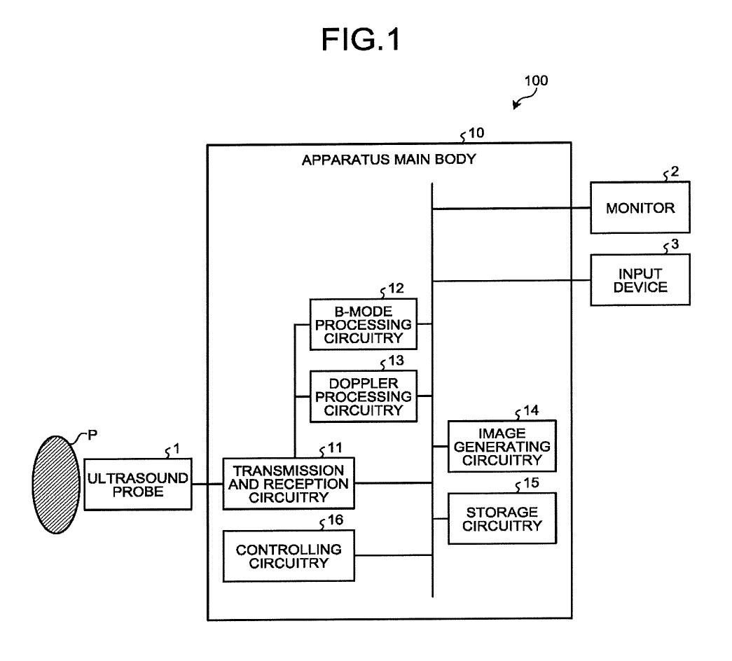

To start with, an exemplary configuration of an ultrasound diagnosis apparatus to which an ultrasound probe according to a first embodiment is applied will be explained. FIG. 1 is a diagram for explaining an exemplary configuration of an ultrasound diagnosis apparatus 100 according to the first embodiment. As illustrated in FIG. 1, the ultrasound diagnosis apparatus 100 according to the first embodiment includes an ultrasound probe 1, a monitor 2, an input device 3, and an apparatus main body 10.

The ultrasound probe 1 is detachably connected to the apparatus main body 10. When an ultrasound wave is transmitted from the ultrasound probe 1 to an examined subject (hereinafter, “patient”) P, the transmitted ultrasound wave is repeatedly reflected on a surface of discontinuity of acoustic impedances at a tissue in the body of the patient P. The reflected ultrasound wave is then received as an echo (a reflected wave) by the ultrasound probe 1. For example, when being a 2D array probe, the ultrasound probe 1 also has large scale integration circuitries therein, so as to perform, together with the apparatus main body 10, controlling and calculation processes on the transmission wave and the reception wave to convert these waves into echo signals. The amplitude of each of the echo signals is dependent on the difference between the acoustic impedances on the surface of discontinuity on which the ultrasound wave is reflected. When a transmitted ultrasound pulse is reflected on the surface of a moving blood flow, a cardiac wall, or the like, the echo signal is, due to the Doppler effect, subject to a frequency shift, depending on a velocity component of the moving members with respect to the ultrasound wave transmission direction.

The monitor 2 is configured to display a Graphical User Interface (GUI) used by a user of the ultrasound diagnosis apparatus 100 for inputting various types of setting requests through the input device 3 and to display an ultrasound image generated by the apparatus main body 10 and the like.

The input device 3 is realized with a trackball, a switch, a dial, a touch command screen, a foot switch, a joystick, and/or the like. The input device 3 is configured to receive the various types of setting requests from the user of the ultrasound diagnosis apparatus 100 and to transfer the received various types of setting requests to the apparatus main body 10. For example, the input device 3 is configured to receive various types of setting requests for controlling the ultrasound probe 1 and to transfer the received various types of setting requests to controlling circuitry 16.

The apparatus main body 10 is an apparatus configured to control the transmission and the reception of the ultrasound waves by the ultrasound probe 1 and is configured to generate an ultrasound image on the basis of the echo signals based on the echo received by the ultrasound probe 1. As illustrated in FIG. 1, the apparatus main body 10 includes transmission and reception circuitry 11, B-mode processing circuitry 12, Doppler processing circuitry 13, mage generating circuitry 14, storage circuitry 15, and the controlling circuitry 16.

The transmission and reception circuitry 11 is configured to transmit and receive various types of data between the ultrasound probe 1 and the apparatus main body 10, under control of the controlling circuitry 16. For example, the transmission and reception circuitry 11 includes an Analog/Digital (A/D) converter and a reception beam former. When the transmission and reception circuitry 11 has received echo signal each of which corresponds to a different one of sub arrays and which were output from the ultrasound probe 1, the A/D converter at first converts the echo signals into pieces of digital data. The reception beam former generate echo data by performing a phased addition process on the pieces of digital data corresponding to the sub arrays and further transmits the generated pieces of echo data to the B-mode processing circuitry 12 and to the Doppler processing circuitry 13.

Further, the transmission and reception circuitry 11 is configured to transmit, to the ultrasound probe 1, the values of the amplitude of drive signals. More specifically, to controlling circuitries 31 (explained later), the transmission and reception circuit 11 transmits the value of the amplitude of each of the drive signals output by the transmission and reception circuitries 22b and 23b (explained later).

Further, the transmission and reception circuitry 11 is configured to transmit, to the ultrasound probe 1, a delay amount for the drive signals, opening control information, a transmission and reception operation mode and timing thereof, and a delay amount for the echo signals received by the ultrasound probe 1.

The B-mode processing circuitry 12 is configured to receive the echo data output from the transmission and reception circuitry 11. Further, the B-mode processing circuitry 12 is configured to generate data (B-mode data) in which signal intensities are expressed by degrees of brightness, by performing a logarithmic amplification process, an envelope detection process, and/or the like on the received echo data. The B-mode processing circuitry 12 may be realized with a processor, for example.

The Doppler processing circuitry 13 is configured to receive the echo data output from the transmission and reception circuitry 11. Further, the Doppler processing circuitry 13 is configured to generate data (Doppler data) obtained by extracting moving member information such as average velocity, dispersion, power, and the like with respect to multiple points, by performing a frequency analysis to obtain velocity information from the received echo data and extracting blood flows, tissues, contrast agent echo components based on the Doppler effect. The Doppler processing circuitry 13 may be realized with a processor, for example.

The image generating circuitry 14 is configured to generate ultrasound images from the data generated by the B-mode processing circuitry 12 and the Doppler processing circuitry 13. In other words, the image generating circuitry 14 is configured to generate a B-mode image in which intensities of the echo are expressed with degrees of brightness, from the B-mode data generated by the B-mode processing circuitry 12. Further, the image generating circuitry 14 is configured to generate an average velocity image, a dispersion image, a power image, or a color Doppler image combining these types of images each of which expresses the moving member information, from the Doppler data generated by the Doppler processing circuitry 13. The image generating circuitry 14 may be realized with a processor, for example.

The storage circuitry 15 may be realized, for example, with a semiconductor memory element such as a Random Access Memory (RAM), a flash memory, or the like, or a hard disk, an optical disk, or the like. For example, the storage circuitry 15 is configured to store therein any of the ultrasound images generated by the image generating circuitry 14. Further, the storage circuitry 15 may store therein any of the data generated by the B-mode processing circuitry 12 and the Doppler processing circuitry 13.

Further, the storage circuitry 15 is configured to store therein control computer programs (hereinafter, “control programs”) for performing ultrasound wave transmissions and receptions, image processing processes, and display processes as well as various types of data such as diagnosis information (e.g., patient's IDs, medical doctors' observations), diagnosis protocols, various types of body marks, and the like.

The controlling circuitry 16 is configured to control overall processes performed by the ultrasound diagnosis apparatus 100. For example, the controlling circuitry 16 is configured to control processes performed by the large scale integration circuitries provided in the ultrasound probe 1, the transmission and reception circuitry 11, the B-mode processing circuitry 12, the Doppler processing circuitry 13, and the image generating circuitry 14, on the basis of the various types of setting requests input by an operator via the input device 3 and any of the various types of control programs and the various types of data read from the storage circuitry 15. Further, the controlling circuitry 16 is configured to control the monitor 2 so as to display any of the ultrasound images stored in the storage circuitry 15, any of the various types of images stored in the storage circuitry 15, a GUI used for implementing processes performed by the image generating circuitry 14, processing results obtained by the image generating circuitry 14, or the like.

For example, the controlling circuitry 16 is configured to control the transmission and reception circuitry 11 so as to transmit the values of the amplitude of the drive signals to the ultrasound probe 1. More specifically, the controlling circuitry 16 is configured to control the transmission and reception circuitry 11 so as to output the value of the amplitude of each of the drive signals, to the controlling circuitries 31 (explained later).

Further, the controlling circuitry 16 is configured to control the transmission and reception circuitry 11 so as to transmit, to the ultrasound probe 1, the delay amount for the drive signals and the delay amount for the echo signals. For example, the controlling circuitry 16 may be realized with a processor.

The term “processor” used in the explanation above denotes, for example, a Central Processing Unit (CPU), a Graphics Processing Unit (GPU), or circuitry such as an Application Specific Integrated Circuit (ASIC) or a programmable logic device (e.g., a Simple Programmable Logic Device [SPLD], a Complex Programmable Logic Device [CPLD], or a Field Programmable Gate Array [FPGA]). One or more processors realize the functions by reading the programs stored in the storage circuitry 15 and executing the read programs. Further, instead of saving the programs in the storage circuitry 15, it is also acceptable to directly incorporate the programs in the circuitries of the processors. In that situation, the processors realize the functions by reading the programs incorporated in the circuitries thereof and executing the read programs. Each of the processors according to the present embodiment does not necessarily have to be structured as single circuitry. It is also acceptable to structure one processor by combining together a plurality of independent circuitries, so as to realize the functions thereof.

An exemplary configuration of the ultrasound diagnosis apparatus 100 according to the first embodiment has thus been explained. As explained below, the ultrasound probe 1 according to the first embodiment is configured to ensure that sufficient areas (circuitry areas) are provided for the electronic circuitries installed within the ultrasound probe 1.

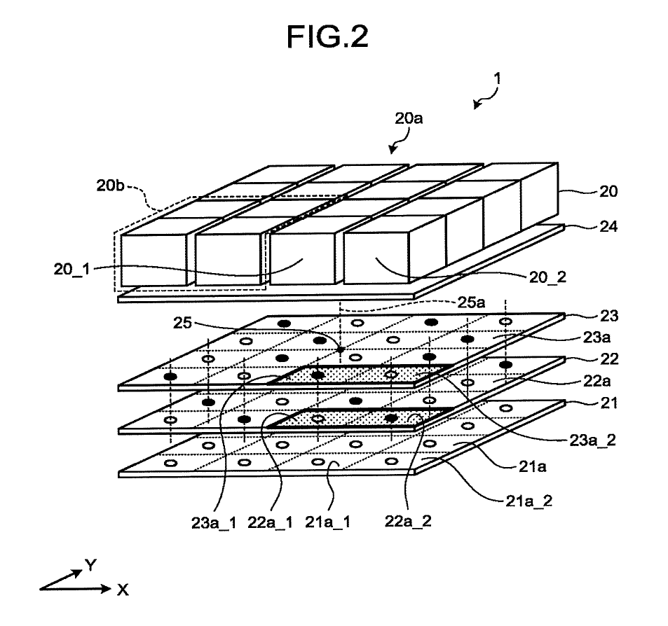

FIG. 2 is a drawing illustrating an exemplary configuration of the ultrasound probe 1 according to the first embodiment. As illustrated in FIG. 2, the ultrasound probe 1 includes a plurality of transducer elements (acoustic elements) 20, three large scale integration circuitries 21 to 23, and a relay substrate 24.

Each of the plurality of transducer elements 20 is configured to transmit an ultrasound wave and to also receive an echo of the ultrasound wave.

The plurality of transducer elements 20 are arranged two-dimensionally. For example, the plurality of transducer elements 20 are arranged along an X-axis and a Y-axis orthogonal to the X-axis. In this situation, the X-axis direction corresponds to a lateral direction, whereas the Y-axis direction corresponds to an elevation direction. In other words, the plurality of transducer elements 20 are arranged in the lateral direction and the elevation direction.

Each of the transducer elements 20 is configured to generate the ultrasound wave on the basis of a drive signal supplied thereto. Further, each of the transducer elements 20 is configured to receive the echo coming from the patient P and to convert the received echo into an echo signal (a reflected-wave signal), which is an electrical signal.

For example, the plurality of transducer elements 20 illustrated in FIG. 2 structure a main array 20a. FIG. 2 illustrates an example in which sixteen transducer elements 20 structure the main array 20a; however, the number of transducer elements 20 structuring the main array 20a is not limited to that in this example. The main array 20a is divided into a plurality of sub arrays 20b in the X-axis direction and the Y-axis direction. For example, when the plurality of transducer elements 20 are organized into groups each having a predetermined number of transducer elements 20, the sub arrays 20b denote positional arrangements of the transducer elements 20 belonging to each of the groups. Each of the sub arrays 20b is structured with the predetermined number of transducer elements 20 that are arranged two-dimensionally in the X-axis direction and the Y-axis direction. For example, the number of transducer elements 20 structuring each of the plurality of sub arrays 20b is four (2×2).

In the first embodiment, the plurality of transducer elements 20 are separated as transducer elements 20 organized in a first group and transducer elements 20 organized in a second group. The transducer elements 20 organized in the first group and the transducer elements 20 organized in the second group will be explained later.

As illustrated in FIG. 2, the three large scale integration circuitries 21 to 23 are arranged in the order of: the large scale integration circuitry 23, the large scale integration circuitry 22, and the large scale integration circuitry 21, starting from the transducer elements 20 side. In other words, the large scale integration circuitry 23 is disposed on the rear face side of the plurality of transducer elements 20. Further, the large scale integration circuitry 22 is disposed on the rear face side of the large scale integration circuitry 23. Further, the large scale integration circuitry 21 is disposed on the rear face side of the large scale integration circuitry 22. The large scale integration circuitry 23 is an example of the first circuitry. The large scale integration circuitry 22 is an example of the second circuitry.

In the large scale integration circuitry 21, each of regions 21a separated by broken lines is a region corresponding to the one transducer element 20 positioned immediately above. Similarly, in the large scale integration circuitries 22 and 23, each of regions 22a and 23a separated by broken lines is also a region corresponding to the one transducer element 20 positioned immediately above.

For example, a region 21a_1 illustrated in FIG. 2 is a region corresponding to the transducer element 20_1 positioned immediately above. In the present example in FIG. 2, among the sixteen (4×4) regions 21a, the region 21a_1 is the third region from the left side in the X-axis direction and the first region from the viewer side in the Y-axis direction. Further, among the sixteen (4×4) transducer elements 20, the transducer element 20_1 is the third transducer element from the left side in the Y-axis direction and the first transducer element from the viewer side in the Y-axis direction.

Further, a region 21a_2 illustrated in FIG. 2 is a region corresponding to the transducer element 20_2 positioned immediately above. In the present example in FIG. 2, among the sixteen regions 21a, the region 21a_2 is the fourth region from the left side in the X-axis direction and the first region from the viewer side in the Y-axis direction. Further, among the sixteen transducer elements 20, the transducer element 20_2 is the fourth transducer element from the left side in the X-axis direction and the first transducer element from the viewer side in the Y-axis direction.

Similarly, a region 22a_1 illustrated in FIG. 2 is a region corresponding to the transducer element 20_1 positioned immediately above. In the present example in FIG. 2, among the sixteen (4×4) regions 22a, the region 22a_1 is the third region from the left side in the X-axis direction and the first region from the viewer side in the Y-axis direction.

Further, a region 22a_2 illustrated in FIG. 2 is a region corresponding to the transducer element 20_2 positioned immediately above. In the present example in FIG. 2, among the sixteen regions 22a, the region 22a_2 is the fourth region from the left side in the X-axis direction and the first region from the viewer side in the Y-axis direction.

Further, a region 23a_1 illustrated in FIG. 2 is a region corresponding to the transducer element 20_1 positioned immediately above. In the present example in FIG. 2, among the sixteen (4×4) regions 23a, the region 23a_1 is the third region from the left side in the X-axis direction and the first region from the viewer side in the Y-axis direction.

Further, a region 23a_2 illustrated in FIG. 2 is a region corresponding to the transducer element 20_2 positioned immediately above. In the present example in FIG. 2, among the sixteen regions 23a, the region 23a_2 is the fourth region from the left side in the X-axis direction and the first region from the viewer side in the Y-axis direction.

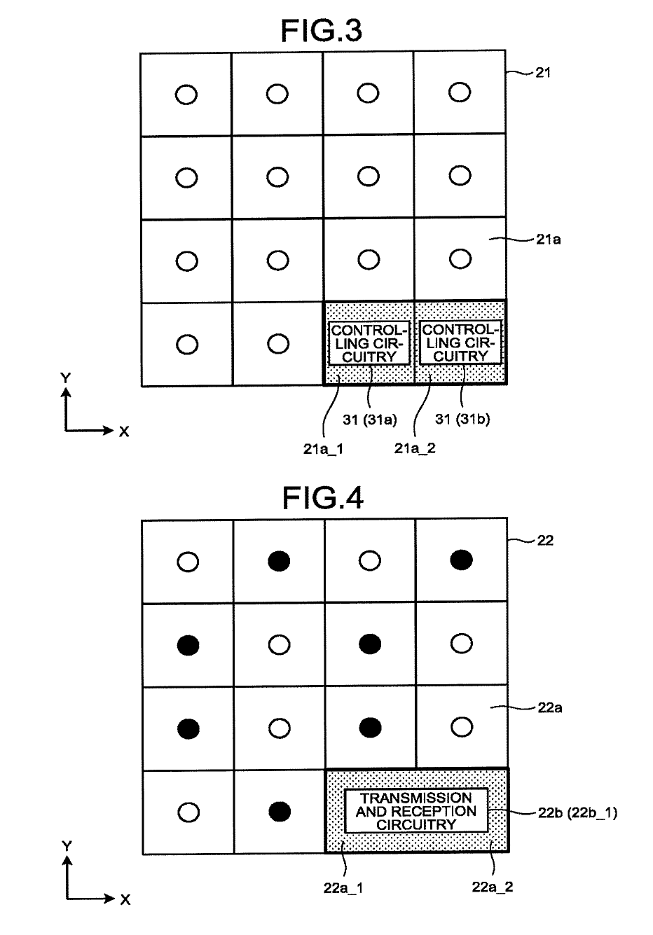

Next, the large scale integration circuitry 21 will be explained. In each of the regions 21a of the large scale integration circuitry 21, controlling circuitry is provided. FIG. 3 is a drawing for explaining an example of the controlling circuitries 31 according to the first embodiment. As illustrated in FIG. 3, for example, provided in the region 21a_1 is controlling circuitry 31 configured to control transmission and reception circuitry 23b_1 (explained later) positioned immediately above. Further, provided in the region 21a_2 is controlling circuitry 31 configured to control transmission and reception circuitry 22b_1 (explained later) positioned immediately above.

The controlling circuitry 31 provided in the region 21a_1 will be referred to as “controlling circuitry 31a”. Further, the controlling circuitry 31 provided in the region 21a_2 will be referred to as “controlling circuitry 31b”.

Further, in each of the other regions 21a (the other fourteen regions 21a), controlling circuitry 31 configured to control either transmission and reception circuitry 22b or transmission and reception circuitry 23b (explained later) positioned immediately above is provided.

Further, in the first embodiment, one channel is assigned to each of the transducer elements 20. For each of the channels, one controlling circuitry 31 and one transmission and reception circuitry 22b or 23b (explained later) are provided. Further, in the first embodiment, in each of the sub arrays 20b, a plurality of (four in FIG. 2) corresponding controlling circuitries 31 are connected together.

For example, each of the controlling circuitries 31 includes a register. When having received the value of the amplitude of a drive signal output from the transmission and reception circuitry 11, each of the controlling circuitries 31 stores the received amplitude value into the register. Further, when having received the delay amount for the drive signal output from the transmission and reception circuitry 11, each of the controlling circuitries 31 stores the received delay amount for the drive signal into the register. Also, when having received the delay amount for the echo signal output from the transmission and reception circuitry 11, each of the controlling circuitries 31 stores the received delay amount for the echo signal into the register.

Further, each of the controlling circuitries 31 is configured to read the amplitude value, the delay amount for the drive signal, and the delay amount for the echo signal, from the register. Further, each of the controlling circuitries 31 is configured to output a control signal indicating the amplitude value, the delay amount for the drive signal, and the delay amount for the echo signal that were read, to corresponding transmission and reception circuitry 22b or 23b.

In this situation, among the plurality of controlling circuitries 31 corresponding to each of the sub arrays 20b, one controlling circuitry 31 includes an adder (a reception beam former). The controlling circuitry 31 including the adder may be referred to as representative controlling circuitry 31.

The adder is configured to acquire a plurality of echo signals output from a plurality of transmission and reception circuitries 22b or 23b (explained later) corresponding to the one sub array 20b. The plurality of echo signals acquired by the adder are echo signals on which a delay process has been performed. Further, the adder performs an adding process to add the plurality of echo signals together. After that, the adder outputs the echo signals resulting from the adding process to the apparatus main body 10. As a result of the adding process using the echo signals, reflection components from the directions corresponding to reception directionality of the echo signals are emphasized. Because this function in the ultrasound probe 1 is implemented on the channels in each of the sub arrays 20b, this function may be referred to as a sub array beam former.

In FIGS. 2 and 3, the white dots depicted in the regions 21a schematically represent conductors each connecting the controlling circuitry 31 provided in the region 21a to the large scale integration circuitry 22. In the first embodiment, for example, terminals or the like may be used as the conductors. In that situation, the controlling circuitry 31 provided in each of the regions 21a is connected to the large scale integration circuitry 22 via the terminal or the like.

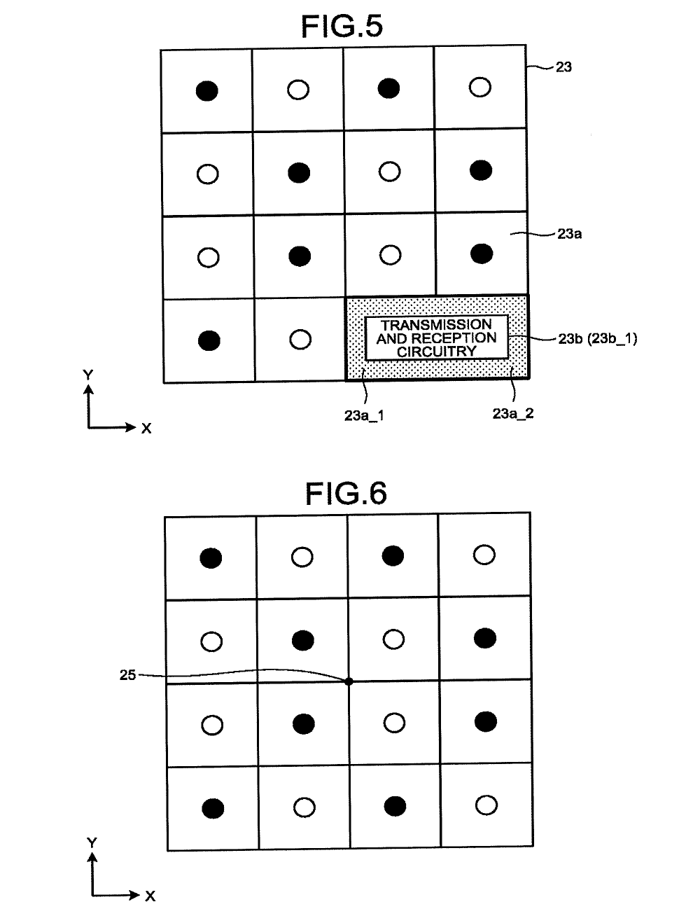

In the large scale integration circuitry 22, each transmission and reception circuitry is provided to extend across a predetermined number of regions 22a. FIG. 4 is a drawing for explaining an example of the transmission and reception circuitry 22b according to the first embodiment. As illustrated in FIG. 4, for example, the transmission and reception circuitry 22b is provided to extend across the two regions 22a_1 and 22a_2 positioned adjacent to each other in the X-axis direction. The transmission and reception circuitry 22b provided to extend across the region 22a_1 and the region 22a_2 will be referred to as “transmission and reception circuitry 22b_1”. The transmission and reception circuitry 22b_1 is configured to drive the transducer element 20_2 and to receive the echo signal from the transducer element 20_2. The transmission and reception circuitry 22b is an example of the electronic circuitry.

In the large scale integration circuitry 22, also with respect to each of the other regions 22a, transmission and reception circuitry 22b is similarly provided to extend across each set of two regions 22a positioned adjacent to each other in the X-axis direction. Each of the transmission and reception circuitries 22b is configured to drive the corresponding one of the transducer elements 20 and to receive the echo signal from the one transducer element 20. In the large scale integration circuitry 22 illustrated in FIGS. 2 and 4, among the sixteen transducer elements 20, eight transmission and reception circuitry 22b corresponding to eight transducer elements 20 are provided.

Each of the transmission and reception circuitries 22b includes analog circuitry such as pulser circuitry, delay circuitry, an amplifier, and/or the like. The pulse circuitry is configured to repeatedly generate a rate pulse used for forming a transmission ultrasound wave at a predetermined rate frequency (called a Pulse Repetition Frequency [PRF]). Further, the pulser circuitry outputs the generated rate pulse as the drive signal for driving the transducer element 20. In this situation, the magnitude of the amplitude of the drive signal output from the pulser circuitry is indicated by the control signal output from the controlling circuitry 31.

The delay circuitry has a function of performing a predetermined delay process on the drive signal output from the pulser circuitry and outputting the drive signal on which the predetermined delay process has been performed to the transducer element 20. For example, under control of the controlling circuitry 31, the delay circuitry performs a delay process of applying a delay amount that is required to converge the ultrasound wave generated from the transducer element 20 into the form of a beam and to determine transmission directionality and that corresponds to the particular transducer element 20, to the drive signal supplied thereto from the pulser circuitry. In this situation, the delay amount to be applied to the drive signal is indicated by the control signal output from the controlling circuitry 31.

The pulser circuitry and the delay circuitry are circuitries configured to drive at least one of the transducer elements 20. The pulser circuitry and the delay circuitry serve as an example of the driving circuitry.

The amplifier may be, for example, an amplifying device. The amplifier has a function of, when having received the echo signal from the transducer element 20, amplifying the received echo signal with a gain that is set in advance and further outputting the amplified echo signal to the delay circuitry.

In this situation, in addition to the function described above, the delay circuitry also has a function of, when having received the echo signal output from the amplifier, performing a delay process of applying a delay amount that is required to determine reception directionality to the received echo signal and further outputting the echo signal on which the delay process has been performed to the controlling circuitry 31. In this situation, the delay amount applied to the echo signal is the delay amount indicated by the control signal output from the controlling circuitry 31.

The amplifier and the delay circuitry are configured to process the echo signal generated by at least one of the transducer elements 20. The amplifier and the delay circuitry serve as an example of the signal processing circuitry. In other words, the delay circuitry is an example of the driving circuitry and is also an example of the signal processing circuitry.

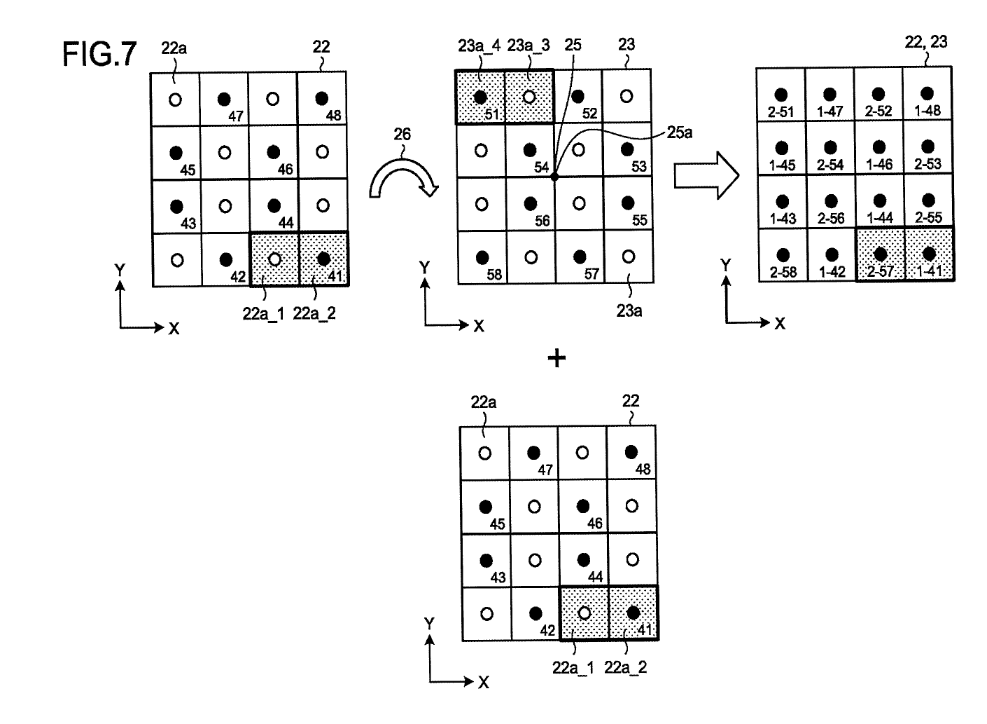

Provided in the large scale integration circuitry 23 are eight transmission and reception circuitries corresponding to the remaining eight transducer elements 20 (the transducer elements 20 that are not connected to the transmission and reception circuitries 22b). In the large scale integration circuitry 23, each of the transmission and reception circuitries is provided to extend across a predetermined number of regions 23a. FIG. 5 is a drawing for explaining an example of the transmission and reception circuitry 23b according to the first embodiment. As illustrated in FIG. 5, for example, the transmission and reception circuitry 23b is provided to extend across the two regions 23a_1 and 23a_2 positioned adjacent to each other in the X-axis direction. The transmission and reception circuitry 23b is an example of the electronic circuitry.

The transmission and reception circuitry 23b provided to extend across the region 23a_1 and the region 23a_2 will be referred to as “transmission and reception circuitry 23b_1”. The transmission and reception circuitry 23b_1 is configured to drive the transducer element 20_1 and to receive the echo signal from the transducer element 20_1.

The transmission and reception circuitry 23b includes analog circuitries similar to the analog circuitries included in the transmission and reception circuitry 22b described above, such as pulser circuitry, delay circuitry, an amplifier, and/or the like.

In the large scale integration circuitry 23, in each of the other regions 23a also, transmission and reception circuitry 23b is similarly provided to extend across each set of two regions 23a positioned adjacent to each other in the X-axis direction. Each of the transmission and reception circuitries 23b is configured to drive the corresponding one of the transducer elements 20 and to receive the echo signal from the one transducer element 20.

Further, in FIGS. 2 and 4, the white dots depicted in the regions 22a schematically represent, for example, conductors each connecting the large scale integration circuitry 21 to the large scale integration circuitry 23. In the first embodiment, for example, terminals and Through Silicon Vias (TSVs) may be used as the conductors. In that situation, in each of the region 22a marked with the white dots, the large scale integration circuitry 21 and the large scale integration circuitry 23 are connected to each other via the terminal and the TSV, or the like. For example, in each of the regions 22a marked with the white dots, the region 21a positioned immediately below is connected to the region 23a positioned immediately above via the terminal and the TSV, or the like.

Further, in FIGS. 2 and 4, the black dots depicted in the regions 22a schematically represent, for example, conductors each connecting the transmission and reception circuitry 22b provided in the region 22a marked with the black dot to the controlling circuitry 31 positioned immediately below and to the region 23a positioned immediately above. In the first embodiment, terminals and TSVs may be used as the conductors, for example. In that situation, in each of the regions 22a marked with the black dots, the transmission and reception circuitry 22b is connected to the controlling circuitry 31 positioned immediately below via the TSV or the like and is also connected to the region 23a positioned immediately above via the terminal or the like.

Further, in FIGS. 2 and 5, the white dots depicted in the regions 23a schematically represent, for example, conductors each connecting the large scale integration circuitry 22 to the transducer element 20. In the first embodiment, for example, terminals and TSVs or the like may be used as the conductors. In that situation, in each of the regions 23a marked with the white dots, the region 22a positioned immediately below is connected to the transducer element 20 positioned immediately above via the terminal and the TSV, or the like. More specifically, in each of the regions 23a marked with the white dots, the region 22a positioned immediately below is connected to the transducer element 20 positioned immediately above via the relay substrate 24, in addition to via the terminal and the TSV, or the like.

Further, in FIGS. 2 and 5, the black dots depicted in the regions 23a schematically represent, for example, conductors each connecting the transmission and reception circuitry 23b provided in the region 23a marked with the black dot to the region 22a positioned immediately below and to the transducer element 20 positioned immediately above. In the first embodiment, terminals and TSVs or the like are used as the conductors, for example. In that situation, in each of the regions 23a marked with the black dots, the transmission and reception circuitry 23b is connected to the region 22a positioned immediately below via the TSV or the like and is also connected to the transducer element 20 positioned immediately above via the terminal or the like. More specifically, the transmission and reception circuitry 23b is connected to the transducer element 20 positioned immediately above via the relay substrate 24, in addition to via the terminal or the like.

The relay substrate 24 is a substrate configured to relay the connection between the terminals provided in the regions 23a and the transducer elements 20. The relay substrate 24 is provided between the transducer elements 20 and the large scale integration circuitry 23. The relay substrate 24 connects the transducer elements 20 and the large scale integration circuitry 20 to each other via wirings provided in the relay substrate 24. In this situation, by adjusting the pitch between the wirings provided in the relay substrate 24, it is possible to absorb the difference found between the pitch (the interval) arranged between the transmission and reception circuitries 22b and 23b on the X-Y plane and the pitch arranged between the transducer elements 20 on the X-Y plane. By using the relay substrate 24 configured in this manner, it is possible to arrange the pitch between the transducer elements 20 to be smaller than the pitch between the transmission and reception circuitries 22b and 23b. Accordingly, it is possible to arrange the transducer elements 20 on the relay substrate 24 with a high density.

The relay substrate 24 may be realized, for example, by using a silicon substrate or a ceramic substrate. Alternatively, it is also acceptable to use a Flexible Printed Circuit (FPC) having the same or similar functions, in place of the relay substrate 24. Further, it is also acceptable to connect the transducer elements 20 to the terminals provided in the regions 23a, without using the relay substrate 24.

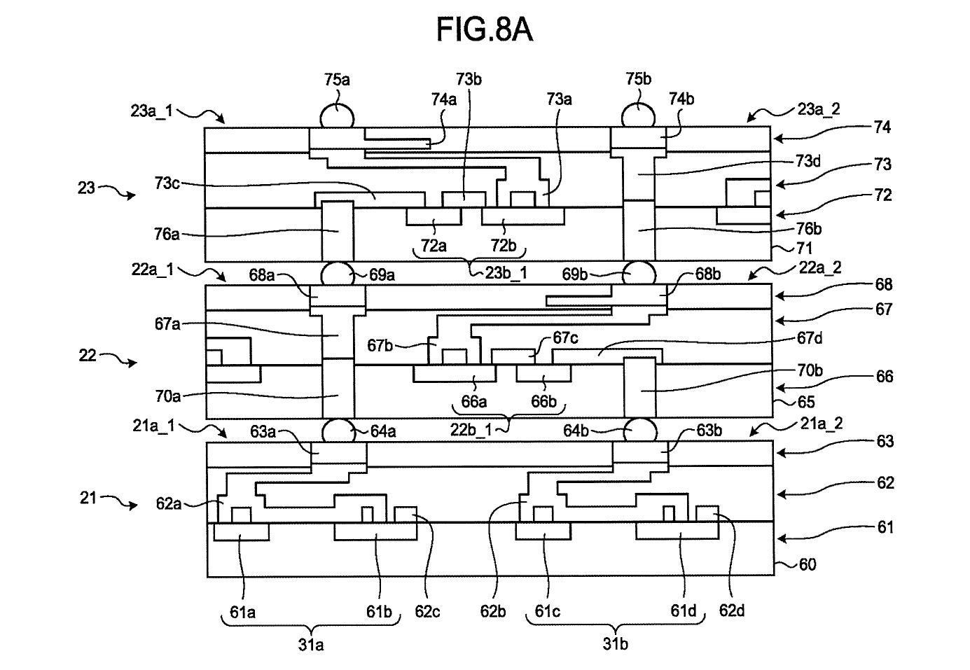

FIG. 6 is a drawing for explaining examples of the two large scale integration circuitries 22 and 23 according to the first embodiment. In this situation, in the first embodiment, the two large scale integration circuitries 22 and 23 are identical large scale integration circuitries. For example, each of the two large scale integration circuitries 22 and 23 is the large scale integration circuitry illustrated in FIG. 6. Each of the large scale integration circuitries 22 and 23 has a center point 25 on the X-Y plane. Further, when each of the large scale integration circuitries 22 and 23 is rotated by 180 degrees on a rotation axis passing through the center point 25 and being orthogonal to the X-axis direction and the Y-axis direction, the black dots and the white dots explained above have a positional relationship as follows: For example, on the X-Y plane, the positions of the black dots after the rotation coincide with the positions of the white dots before the rotation, whereas the positions of the white dots after the rotation coincide with the positions of the black dots before the rotation.

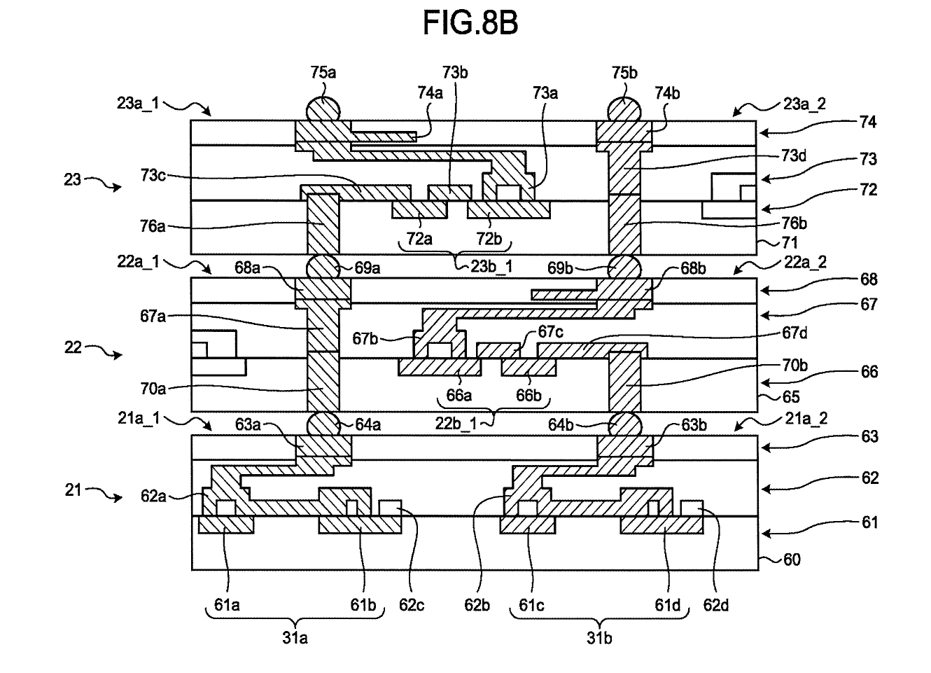

FIG. 7 is a drawing illustrating a part of a method for manufacturing the ultrasound probe 1 according to the first embodiment. In the ultrasound probe 1 according to the first embodiment, the two large scale integration circuitries 22 and 23 are arranged in mutually the same orientation, and subsequently, as illustrated in the middle section of FIG. 7, with respect to the large scale integration circuitry 22, the large scale integration circuitry 23 is rotated by 180 degrees as indicated with an arrow 26, on a rotation axis 25a passing through the center point 25 and being orthogonal to the X-axis direction and the Y-axis direction. After that, the large scale integration circuitry 23 that was rotated by 180 degrees is arranged over the large scale integration circuitry 22.

In other words, the large scale integration circuitry 23 is arranged in a predetermined orientation on the rear face side of the plurality of transducer elements 20, while the large scale integration circuitry 22 is arranged, in an orientation different from the predetermined orientation, on the rear face side of the large scale integration circuitry 23. In this manner, the large scale integration circuitry 22 and the large scale integration circuitry 23 are identical large scale integration circuitry that are arranged in mutually-different orientations. As a result, according to the first embodiment, when manufacturing the large scale integration circuitry 22 and the large scale integration circuitry 23, it is sufficient when identical large scale integration circuitries are manufactured, without the need to manufacture a plurality of types of large scale integration circuitries. It is therefore possible to keep the manufacturing costs low. In this situation, the predetermined orientation is an example of the first orientation, whereas the orientation different from the predetermined orientation is an example of the second orientation.

The reference numerals “41” to “48” in FIG. 7 indicate the black dots depicted in the regions 22a explained above. The reference numerals “51” to “58” indicate the black dots depicted in the regions 23a explained above.

In FIG. 7, the regions 22a_1 and 22a_2 indicated with hatching correspond to the regions 23a 3 and 23a 4 indicated with hatching.

In the right section of FIG. 7, the notation “N-M” in which a numeral N (where N=1 or 2) is joined by a hyphen “-” with another numeral M (where M=41 to 48 and 51 to 58) indicates that, in the large scale integration circuitry expressed by “N”, the region 22a or 23a marked with the black dot expressed by the numeral “M” is provided with a conductor represented by the black dot expressed by the numeral “M”. For example, when “N” is “1”, “N” represents the large scale integration circuitry 22. When “N” is “2”, “N” represents the large scale integration circuitry 23. For example, the notation “2-51” indicates that, in the large scale integration circuitry 23, the region 23a marked with the black dot expressed with the numeral “51” is provided with a conductor such as a TSV connected to the transmission and reception circuitry 23b. The notation “1-47” indicates that, in the large scale integration circuitry 22, the region 22a marked with the black dot expressed with the numeral “47” is provided with a conductor such as a TSV connected to the transmission and reception circuitry 22b.

In the first embodiment, as illustrated in the right section of FIG. 7, each of the regions (the eight regions 22a and the eight regions 23a) corresponding to all the transducer elements 20 is provided with a conductor connected either to the transmission and reception circuitry 22b or to the transmission and reception circuitry 23b. In other words, each of all the transducer elements 20 is connected to one selected from between the transmission and reception circuitry 22b and the transmission and reception circuitry 23b. In another example, all the transducer elements 20 do not necessarily have to be connected to one selected from between the transmission and reception circuitry 22b and the transmission and reception circuitry 23b. For example, it is sufficient when the transmission and reception circuitries 22b are connected to at least one of the plurality of transducer elements 20. Similarly, it is sufficient when the transmission and reception circuitries 23b are connected to at least one of the plurality of transducer elements 20.

In this situation, in the first embodiment, the transducer elements 20 organized in the first group mentioned above are connected to the transmission and reception circuitries 23b, so that the drive signals are supplied thereto from the transmission and reception circuitries 23b. In contrast, the transducer elements 20 organized in the second group mentioned above are connected to the transmission and reception circuitries 22b, so that the drive signals are supplied thereto from the transmission and reception circuitries 22b. In other words, the transducer elements 20 organized in the first group correspond to the transmission and reception circuitries 23b, whereas the transducer elements 20 organized in the second group correspond to the transmission and reception circuitries 22b.

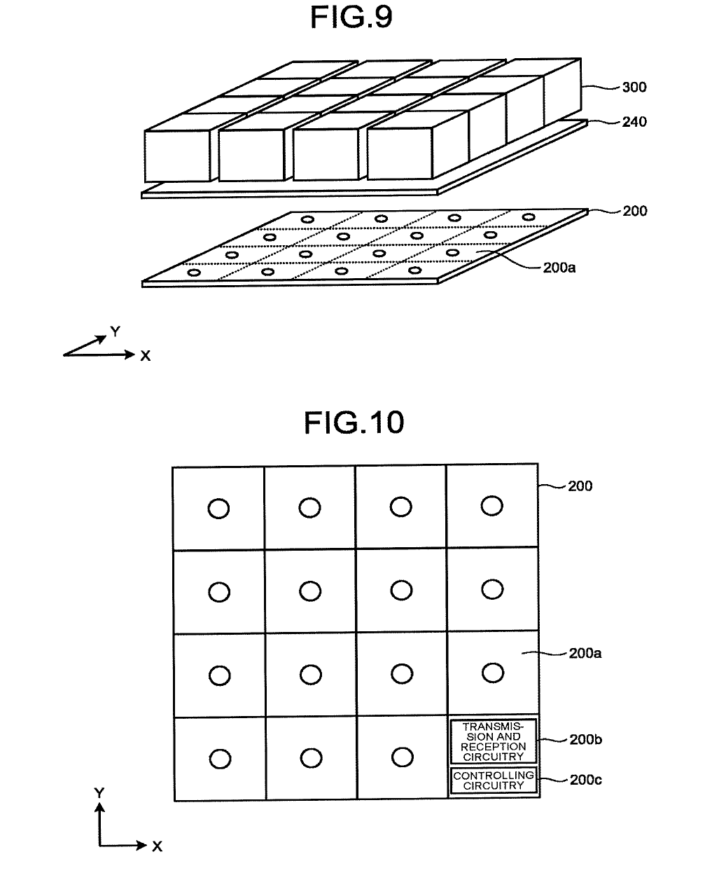

FIG. 8A is a drawing for explaining an example of a laminated structure of the three large scale integration circuitries 21 to 23 according to the first embodiment. FIG. 8A is a cross-sectional view of the part corresponding to the regions 21a_1, 21a_2, 22a_1, 22a_2, 23a_1, and 23a_2 mentioned above.

As illustrated in FIG. 8A, the large scale integration circuitry 21 includes a transistor layer 61, a metal multi-layer wiring layer 62, and a re-wiring layer 63. The transistor layer 61, the metal multi-layer wiring layer 62, and the re-wiring layer 63 are laminated in the stated order. Further, the example illustrated in FIG. 8A is an example of the one cross-sectional plane. In actuality, however, the transistor layer 61, the metal multi-layer wiring layer 62, and the re-wiring layer 63 are also present in the depth direction, and circuitries and wiring necessary for operations are formed.

The transistor layer 61 is formed on a surface of a silicon substrate 60. The transistor layer 61 has transistors 61a to 61d formed therein. The transistors 61a and 61b are elements structuring the controlling circuitry 31a explained above. The transistors 61c and 61d are elements structuring the controlling circuitry 31b explained above.

The metal multi-layer wiring layer 62 is formed to have a laminated structure including a plurality of electrically-insulative layers and a plurality of wirings (of metal). In the explanations below, the plurality of wirings that are laminated will simply be referred to as “wirings”. The metal multi-layer wiring layer 62 has wirings 62a to 62d formed therein. The wiring 62a is connected to the transistor 61a and to the transistor 61b. The wiring 62b is connected to the transistor 61c and to the transistor 61d.

Further, the wiring 62c is connected to the transistor 61b, whereas the wiring 62d is connected to the transistor 61d. Furthermore, although not illustrated in FIG. 8A, in the metal multi-layer wiring layer 62, the wiring 62c and the wiring 62d are connected to each other. As a result, the controlling circuitry 31a and the controlling circuitry 31b are able to transmit and receive the echo signals to and from each other. For example, when the controlling circuitry 31a is the representative controlling circuitry mentioned above, the controlling circuitry 31a is able to receive the echo signals subject to the adding process, from the controlling circuitry 31b.

The re-wiring layer 63 has wirings 63a and 63b formed therein. The wiring 63a is connected to the wiring 62a. The wiring 63b is connected to the wiring 62b.

Each of terminals 64a and 64b is formed by using one selected from among a metal pad, an electrode, and a bump. The terminal 64a is connected to the wiring 63a. The terminal 64b is connected to the wiring 63b.

Accordingly, the transistor 61a and the transistor 61b are connected to the terminal 64a via the wiring 63a and the wiring 62a. Further, the transistor 61c and the transistor 61d are connected to the terminal 64b via the wiring 63b and the wiring 62b. As a result, the controlling circuitry 31a is connected to the terminal 64a, whereas the controlling circuitry 31b is connected to the terminal 64b.

The large scale integration circuitry 21 has the same configuration in the regions 21a other than the regions 21a_1 and 21a_2.

The large scale integration circuitry 22 includes a transistor layer 66, a metal multi-layer wiring layer 67, and a re-wiring layer 68. The transistor layer 66, the metal multi-layer wiring layer 67, and the re-wiring layer 68 are laminated in the stated order.

The transistor layer 66 is formed on a surface of a silicon substrate 65. The transistor layer 66 has transistors 66a and 66b formed therein. The transistors 66a and 66b are elements structuring the transmission and reception circuitry 22b_1 explained above. Further, the example illustrated in FIG. 8A is an example of the one cross-sectional plane. In actuality, however, the transistor layer 66, the metal multi-layer wiring layer 67, and the re-wiring layer 68 are also present in the depth direction, and circuitries and wiring necessary for operations are formed.

Further, the silicon substrate 65 has formed therein a TSV 70a penetrating the silicon substrate 65 in the position corresponding to the terminal 64a. In addition, the silicon substrate 65 has formed therein a TSV 70b penetrating the silicon substrate 65 in the position corresponding to the terminal 64b.

The TSV 70a is connected to the terminal 64a. The TSV 70b is connected to the terminal 64b.

The metal multi-layer wiring layer 67 is formed to have a laminated structure including a plurality of electrically-insulative layers and a plurality of wirings. The metal multi-layer wiring layer 67 has formed therein wirings 67a to 67d. The wiring 67a is connected to the TSV 70a.

The wiring 67b is connected to the transistor 66a. The wiring 67c is connected to the transistor 66a and to the transistor 66b. The wiring 67d is connected to the transistor 66b and to the TSV 70b and is connected as an element that is controllable by the controlling circuitry 31b included in the large scale integration circuitry 21.

The re-wiring layer 68 has formed therein wirings 68a and 68b. The wiring 68a is connected to the wiring 67a. The wiring 68b is connected to the wiring 67b.

Each of terminals 69a and 69b is formed by using one selected from among a metal pad, an electrode, and a bump. The terminal 69a is connected to the wiring 68a. The terminal 69b is connected to the wiring 68b.

As a result, the terminal 69a is connected to the terminal 64a via the wiring 68a, the wiring 67a, and the TSV 70a and is thus connected to the controlling circuitry 31a included in the large scale integration circuitry 21. In contrast, the terminal 69b is connected to the terminal 64b via the wiring 68b, the wiring 67b, the transistor 66a, the wiring 67c, the transistor 66b, the wiring 67d, and the TSV 70b. Accordingly, the transmission and reception circuitry 22b_1 is connected to the terminal 69b and to the terminal 64b.

The large scale integration circuitry 22 has the same configuration in the regions 22a other than the regions 22a_1 and 22a_2.

The large scale integration circuitry 23 includes a transistor layer 72, a metal multi-layer wiring layer 73, and a re-wiring layer 74. The transistor layer 72, the metal multi-layer wiring layer 73, and the re-wiring layer 74 are laminated in the stated order.

The transistor layer 72 is formed on a surface of a silicon substrate 71. The transistor layer 72 has transistors 72a and 72b formed therein. The transistors 72a and 72b are elements structuring the transmission and reception circuitry 23b_1 explained above. Further, the example illustrated in FIG. 8A is an example of the one cross-sectional plane. In actuality, however, the transistor layer 72, the metal multi-layer wiring layer 73, and the re-wiring layer 74 are also present in the depth direction, and circuitries and wiring necessary for operations are formed.

Further, the silicon substrate 71 has formed therein a TSV 76a penetrating the silicon substrate 71 in the position corresponding to the terminal 69a. In addition, the silicon substrate 71 has formed therein a TSV 76b penetrating the silicon substrate 71 in the position corresponding to the terminal 69b.

The TSV 76a is connected to the terminal 69a. The TSV 76b is connected to the terminal 69b.

The metal multi-layer wiring layer 73 is formed to have a laminated structure including a plurality of electrically-insulative layers and a plurality of wirings. The metal multi-layer wiring layer 73 has formed therein wirings 73a to 73d.

The wiring 73a is connected to the transistor 72b. The wiring 73b is connected to the transistor 72a and to the transistor 72b. The wiring 73c is connected to the transistor 72a and to the TSV 76a and is connected as an element that is controllable by the controlling circuitry 31a included in the large scale integration circuitry 21. The wiring 73d is connected to the TSV 76b.

The re-wiring layer 74 has wirings 74a and 74b formed therein. The wiring 74a is connected to the wiring 73a. The wiring 74b is connected to the wiring 73d.

Each of terminals 75a and 75b is formed by using one selected from among a metal pad, an electrode, and a bump. The terminal 75a is connected to the wiring 74a. The terminal 75b is connected to the wiring 74b.

As a result, the terminal 75a is connected to the terminal 69a via the wiring 74a, the wiring 73a, the transistor 72b, the wiring 73b, the transistor 72a, the wiring 73c, and the TSV 76a. Accordingly, the transmission and reception circuitry 23b_1 is connected to the terminal 75a and to the terminal 69a. Further, the terminal 75b is connected to the terminal 69b, via the wiring 74a, the wiring 73d, and the TSV 76b.

The terminal 75a is connected to the transducer element 20_1 via the relay substrate 24. Further, the terminal 75b is connected to the transducer element 20_2 via the relay substrate 24.

The large scale integration circuitry 23 has the same configuration in the regions 23a other than the regions 23a_1 and 23a_2.

FIG. 8B is a drawing for clearly illustrating connection relationships in the example in FIG. 8A. In the configuration described above, as illustrated in the part (a first part) indicated with the hatching lines extending from the top left to the bottom right in FIG. 8B, the transmission and reception circuitry 23b_1 is connected to the transducer element 20_1 (see FIG. 2). For example, the transmission and reception circuitry 23b_1 outputs the drive signal to the transducer element 20_1 via the wiring 73a, the wiring 74a, the terminal 75a, and the relay substrate 24 (see FIG. 2). Further, the transducer element 20_1 outputs the echo signal to the transmission and reception circuitry 23b_1 via the relay substrate 24, the terminal 75a, the wiring 74a, and the wiring 73a.

Also, as illustrated in the first part, the controlling circuitry 31a is connected to the transmission and reception circuitry 23b_1. For example, the controlling circuitry 31a outputs the control signal to the transmission and reception circuitry 23b_1, via the wiring 62a, the wiring 63a, the terminal 64a, the TSV 70a, the wiring 67a, the wiring 68a, the terminal 69a, the TSV 76a, and the wiring 73c. Further, the transmission and reception circuitry 23b_1 outputs the echo signal to the controlling circuitry 31a, via the wiring 73c, the TSV 76a, the terminal 69a, the wiring 68a, the wiring 67a, the TSV 70a, the terminal 64a, the wiring 63a, and the wiring 62a.

Further, as illustrated in the part (a second part) indicated with the hatching lines extending from the top right to the bottom left in FIG. 8B, the transmission and reception circuitry 22b_1 is connected to the transducer element 20_2 (see FIG. 2). For example, the transmission and reception circuitry 22b_1 outputs the drive signal to the transducer element 20_2 via the wiring 67b, the wiring 68b, the terminal 69b, the TSV 76b, the wiring 73d, the wiring 74b, the terminal 75b, and the relay substrate 24. Further, the transducer element 20_2 outputs the echo signal to the transmission and reception circuitry 22b_1 via the relay substrate 24, the terminal 75b, the wiring 74b, the wiring 73d, the TSV 76b, the terminal 69b, the wiring 68b, and the wiring 67b.

Also, as illustrated in the second part, the controlling circuitry 31b is connected to the transmission and reception circuitry 22b_1. For example, the controlling circuitry 31b outputs the control signal to the transmission and reception circuitry 22b_1, via the wiring 62b, the wiring 63b, the terminal 64b, the TSV 70b, and the wiring 67d. Further, transmission and reception circuitry 22b_1 outputs the echo signal to the controlling circuitry 31b, via the wiring 67d, the TSV 70b, the terminal 64b, the wiring 63b, and the wiring 62b.

In this situation, the terminal 64a and the wirings 63a and 62a as well as the terminal 64b and the wirings 63b and 62b correspond to white dots depicted in the regions 21a in FIGS. 2 and 3.

Further, the terminal 69a, the wiring 68a, the wiring 67a, and the TSV 70a correspond to a white dot depicted in the region 22a in FIGS. 2 and 4. Further, the terminal 69b, the wiring 68b, the wiring 67b, the wiring 67d, and the TSV 70b correspond to a black dot depicted in the region 22a in FIGS. 2 and 4.

Further, the terminal 75a, the wiring 74a, the wiring 73a, the wiring 73c, and the TSV 76a correspond to a black dot depicted in the region 23a in FIGS. 2 and 5. Further, the terminal 75b, the wiring 74b, the wiring 73d, and the TSV 76b correspond to a white dot depicted in the region 23a in FIGS. 2 and 5.

As explained above, the large scale integration circuitry 22 is provided with TSVs (the TSV 70a in FIG. 8B) corresponding to the transducer elements 20 organized in the first group explained above and is provided with the transmission and reception circuitries 22b (the transmission and reception circuitry 22b_1 in FIG. 8B) corresponding to the transducer elements 20 organized in the second group. The TSVs corresponding to the transducer elements 20 organized in the first group are connected to the transmission and reception circuitries 23b provided in the large scale integration circuitry 23. By using this configuration, it is possible to ensure that the area equal to two of the regions 22a is provided as the circuitry area of each of the transmission and reception circuitries 22b provided for the large scale integration circuitry 22. In other words, it is possible to ensure that the area equal to two of the regions 22a is provided as the circuitry area of each of the transmission and reception circuitries 22b corresponding to a single transducer element 20.

Further, the large scale integration circuitry 23 is provided with the transmission and reception circuitries 23b (the transmission and reception circuitry 23b_1 in FIG. 8B) corresponding to the transducer elements 20 organized in the first group and is provided with the TSVs (the TSV 76b in FIG. 8B) corresponding to the transducer elements 20 organized in the second group. The TSVs corresponding to the transducer elements 20 organized in the second group are connected to the transmission and reception circuitries 22b provided in the large scale integration circuitry 22. By using this configuration, it is possible to ensure that the area equal to two of the regions 23a is provided as the circuitry area of each of the transmission and reception circuitries 23b provided in the large scale integration circuitry 23. In other words, it is possible to ensure that the area equal to two of the regions 23a is provided as the circuitry area of each of the transmission and reception circuitries 23b corresponding to a single transducer element 20.

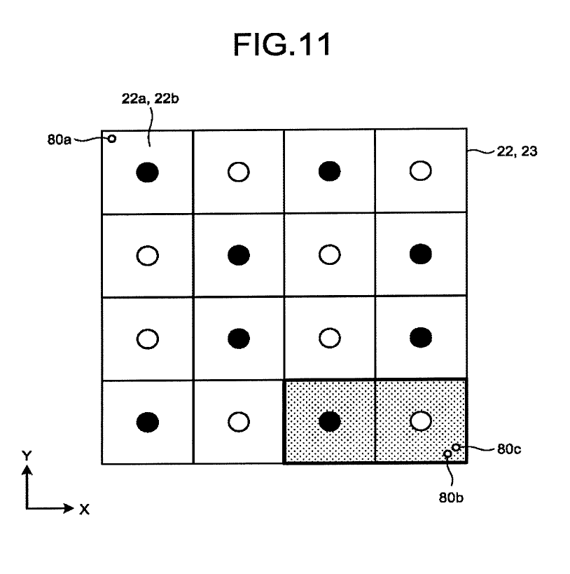

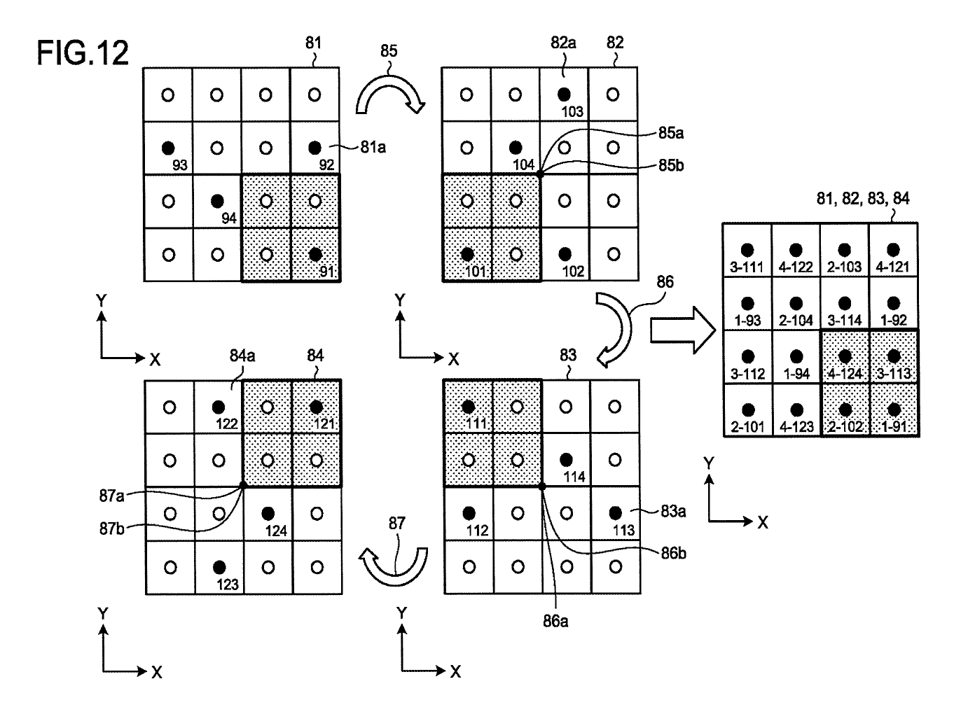

The ultrasound diagnosis apparatus 100 according to the first embodiment has thus been explained. Next, an example will be explained in which transmission and reception circuitry and controlling circuitry are provided in each of the regions in large scale integration circuitry. FIGS. 9 and 10 are drawings illustrating the example in which transmission and reception circuitry and controlling circuitry are provided in each of the regions of the large scale integration circuitry. FIGS. 9 and 10 each illustrate one large scale integration circuitry 200. In each of regions 200a of the large scale integration circuitry 200, transmission and reception circuitry 200b and controlling circuitry 200c are provided, as illustrated in FIG. 10.

Further, the transmission and reception circuitry 200b is connected to a transducer element 300 positioned immediately above, via a relay substrate 240. In that situation, what is provided as the circuitry area of the transmission and reception circuitry 200b is only the area of the one region 200a corresponding to the one transducer element 300. In addition, the transmission and reception circuitry 200b shares the one region 200a with the controlling circuitry 200c. Accordingly, in the example illustrated in FIGS. 9 and 10, it would be difficult to enlarge the size of the area provided as the circuitry area of the transmission and reception circuitry 200b. In other words, it would be difficult to ensure that a sufficient circuitry area is provided for the transmission and reception circuitry 200b.

There are manufacturing processes with which it is possible to make the size (the circuitry area) of electronic circuitries of various types relatively small. Thus, by using one of such manufacturing processes, it might be possible to manufacture the large scale integration circuitry 200 provided with relatively small transmission and reception circuitry 200b. However, for example, the characteristics and performance level of the transmission and reception circuitries 200b included in an ultrasound probe are determined in advance at the stage of designing. In that situation, for the reasons stated below, it would be difficult to reduce the size of the transmission and reception circuitries 200b in each of which analog circuitry is dominant.

For example, the size of the transistor included in the transmission and reception circuitry 200b is determined in accordance with the characteristics and performance level of the transmission and reception circuitry 200b. For this reason, if the size of the transistor was reduced, the characteristics and performance level would be different from those determined at the stage of designing. As explained herein, although there are manufacturing processes with which it is possible to reduce the size of the transmission and reception circuitries 200b, it would be difficult to satisfy the determined characteristics and performance level, if the large scale integration circuitry 200 were manufactured by using one of such manufacturing processes. In other words, in the example illustrated in FIGS. 9 and 10, it would be difficult to ensure that a sufficient circuitry area is provided for the transmission and reception circuitry 200b, the circuitry area being determined in accordance with the designed characteristics and performance level.

To cope with this situation, the ultrasound probe 1 according to the first embodiment ensures that a plurality of (e.g., two) regions 22a or 23a are provided, instead of one region 22a or 23a, as the area for each of the single transmission and reception circuitries 22b and 23b. As a result, according to the first embodiment, it is possible to ensure that a sufficient circuitry area is provided for each of the single transmission and reception circuitries 22b and 23b in correspondence with a single transducer element 20.

The plurality of transducer elements 20 according to the first embodiment may be organized into one group of transducer elements 20 each configured to transmit an ultrasound wave and another group of transducer elements 20 each configured to receive an echo. In other words, it is acceptable when each of the transducer elements 20 is configured to perform at least one selected from between the transmission of the ultrasound wave and the reception of the ultrasound wave.

### First Modification Example of First Embodiment

Next, an ultrasound probe according to a first modification example of the first embodiment will be explained. Some of the constituent elements that are the same as those included in the ultrasound probe 1 according to the first embodiment will be referred to by using the same reference characters, and the explanations thereof may be omitted.

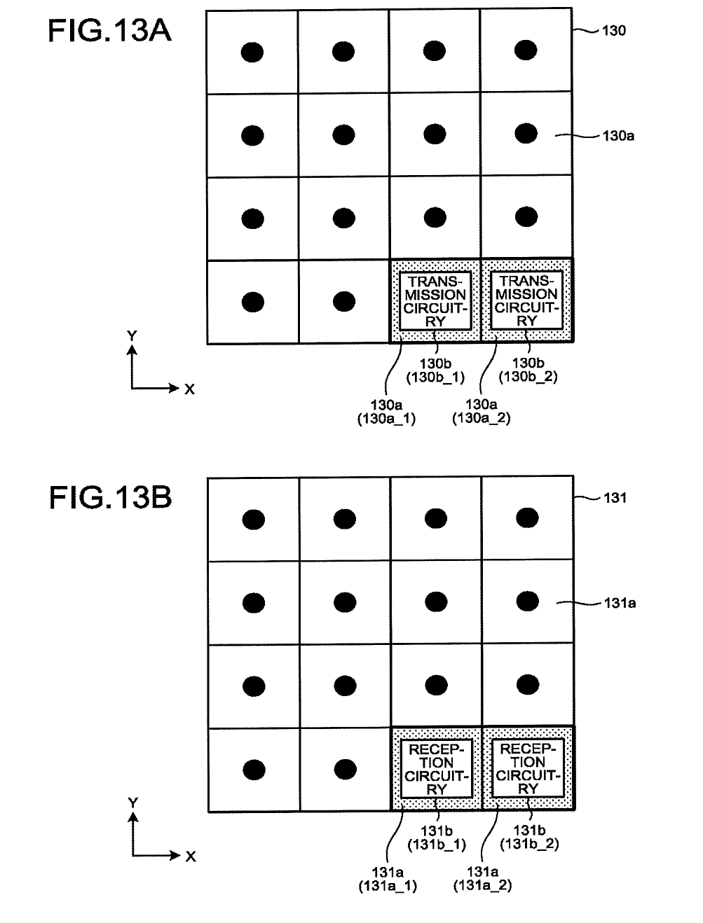

FIG. 11 is a drawing for explaining an example of the ultrasound probe according to the first modification example of the first embodiment. The ultrasound probe according to the first modification example includes the large scale integration circuitries 22 and 23 illustrated in FIG. 11. Each of the large scale integration circuitries 22 and 23 illustrated in FIG. 11 is provided with one marker 80a and two markers 80b and 80c in the diagonal corners. As a result, when manufacturing the ultrasound probe, developers or the like are able to easily understand the orientations of the large scale integration circuitries 22 and 23, by checking the positions of the one marker 80a and the two markers 80b and 80c. Consequently, the first modification example makes it easier for the developers or the like to manufacture the ultrasound probe.

In this situation, any of various materials may be used as the markers 80a, 80b and 80c. For example, it is possible to expose the wirings formed in the re-wiring layers 68 and 74 and to use the exposed wirings as the markers 80a, 80b, and 80c.

### Second Modification Example of First Embodiment

In the first embodiment, the example is explained in which the ultrasound probe 1 includes the two large scale integration circuitries 22 and 23 provided with the transmission and reception circuitries 22b and 23b, respectively. However, the ultrasound probe may include K or more (where K is an integer of 3 or larger) large scale integration circuitries each provided with transmission and reception circuitry. Thus, this modification example will be explained as a second modification example of the first embodiment. Some of the constituent elements that are the same as those included in the ultrasound probe 1 according to the first embodiment may be referred to by using the same reference characters, and the explanations thereof may be omitted. In the following sections, an example in which K=4 will be explained.

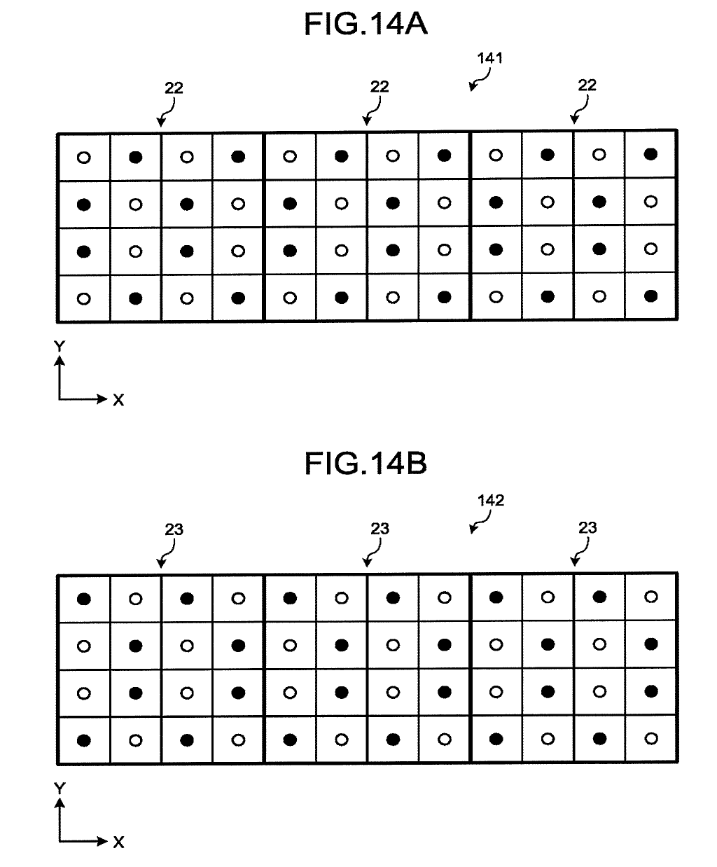

FIG. 12 is a drawing illustrating an example of a part of a method for manufacturing an ultrasound probe according to the second modification example of the first embodiment. As illustrated in FIG. 12, in the second modification example, four large scale integration circuitries 81 to 84 are identical large scale integration circuitries.

The large scale integration circuitries 82 to 84 have center points 85a, 86a, and 87a on the X-Y plane, respectively. Further, the four large scale integration circuitries 81 to 84 are positioned in mutually—the same orientation, and subsequently, with respect to the large scale integration circuitry 81, the large scale integration circuitry 82 is rotated by 90 degrees as indicated by an arrow 85, on a rotation axis 85b passing through the center point 85a and being orthogonal to the X-direction and the Y-direction. In other words, the orientation of the large scale integration circuitry 82 installed in the ultrasound probe is different from that of the large scale integration circuitry 81 by 90 degrees.

Further, with respect to the large scale integration circuitry 81, the large scale integration circuitry 83 is rotated by 180 degrees as indicated by the arrow 85 and another arrow 86, on a rotation axis 86b passing through the center point 86a and being orthogonal to the X-direction and the Y-direction. In other words, the orientation of the large scale integration circuitry 83 installed in the ultrasound probe is different from that of the large scale integration circuitry 81 by 180 degrees.

Further, with respect to the large scale integration circuitry 81, the large scale integration circuitry 84 is rotated by 270 degrees as indicated by the arrows 85 and 86 and another arrow 87, on a rotation axis 87b passing through the center point 87a and being orthogonal to the X-direction and the Y-direction. In other words, the orientation of the large scale integration circuitry 84 installed in the ultrasound probe is different from that of the large scale integration circuitry 81 by 270 degrees.

Further, the large scale integration circuitry 82 that was rotated by 90 degrees is arranged over the large scale integration circuitry 81. The large scale integration circuitry 83 that was rotated by 180 degrees is arranged over the large scale integration circuitry 82. Also, the large scale integration circuitry 84 that was rotated by 270 degrees is arranged over the large scale integration circuitry 83.

In this situation, the black dots and the white dots explained above have a relationship as follows: For example, on the X-Y plane, the positions of three white dots coincide with the position of one black dot.

The numerals “91” to “94” in FIG. 12 indicate the black dots depicted in the regions 81a of the large scale integration circuitry 81. In FIG. 12, the black dots indicated by the numerals “91” to “94” schematically represent conductors that are each provided in the region 81a marked with the black dot and are each connected to transmission and reception circuitry.

In the large scale integration circuitry 81, the transmission and reception circuitry is provided to extend across a region 81a marked with the black dot and three regions (the three regions marked with white dots) 81a positioned adjacent to the region 81a in three directions, namely, the X-axis direction, the Y-axis direction, and a diagonal direction different from the X- and the Y-axis directions. In other words, in the large scale integration circuitry 81, the transmission and reception circuitry is provided to extend across four of the regions 81a.

Further, the numerals “101” to “104” indicate the black dots depicted in the regions 82a of the large scale integration circuitry 82. In FIG. 12, the black dots indicated by the numerals “101” to “104” schematically represent conductors that are each provided in the region 82a marked with the black dot and are each connected to transmission and reception circuitry similarly to the large scale integration circuitry 81, in the large scale integration circuitry 82, the transmission and reception circuitry is provided to extend across four of the regions 82a.

Further, the numerals “111” to “114” indicate the black dots depicted in the regions 83a of the large scale integration circuitry 83. In FIG. 12, the black dots indicated by the numerals “111” to “114” schematically represent conductors that are each provided in the region 83a marked with the black dot and are each connected to transmission and reception circuitry similarly to the large scale integration circuitry 81, in the large scale integration circuitry 83, the transmission and reception circuitry is provided to extend across four of the regions 83a.

Further, the numerals “121” to “124” indicate the black dots depicted in the regions 84a of the large scale integration circuitry 84. In FIG. 12, the black dots indicated by the numerals “121” to “124” schematically represent conductors that are each provided in the region 84a marked with the black dot and are each connected to transmission and reception circuitry similarly to the large scale integration circuitry 81, in the large scale integration circuitry 84, the transmission and reception circuitry is provided to extend across four of the regions 84a.

Further, the white dots depicted in the regions 81a schematically represent conductors that are each provided in the region 81a marked with the white dot and each connecting the controlling circuitry 31 positioned immediately below to the region 82a positioned immediately above. The white dots depicted in the regions 82a schematically represent conductors that are each provided in the region 82a marked with the white dot and each connecting the region 81a positioned immediately below to the region 83a positioned immediately above. The white dots depicted in the regions 83a schematically represent conductors that are each provided in the region 83a marked with the white dot and each connecting the region 82a positioned immediately below to the region 84a positioned immediately above. The white dots depicted in the regions 84a schematically represent conductors that are each provided in the region 84a marked with the white dot and each connecting the region 83a positioned immediately below to the transducer element 20 positioned immediately above.

In FIG. 12, the four regions 81a indicated with the hatching in the large scale integration circuitry 81, the four regions 82a indicated with the hatching in the large scale integration circuitry 82, the four regions 83a indicated with the hatching in the large scale integration circuitry 83, and the four regions 84a indicated with the hatching in the large scale integration circuitry 84 correspond to one another.

In the right section of FIG. 12, the notation “Q-R” in which a numeral Q (where Q=1, 2, 3, and 4) is joined by a hyphen “-” with another numeral R (where R=91 to 94, 101 to 104, 111 to 114, and 121 to 124) indicates that, in the large scale integration circuitry expressed by “Q”, the region 81a, 82a, 83a, or 84a marked with the black dot expressed by the numeral “R” is provided with a conductor represented by the black dot expressed by the numeral “R”. For example, when “Q” is “1”, “Q” represents the large scale integration circuitry 81. When “Q” is “2”, “Q” represents the large scale integration circuitry 82. When “Q” is “3”, “Q” represents the large scale integration circuitry 83. When “Q” is “4”, “Q” represents the large scale integration circuitry 84.

For example, the notation “3-111” indicates that, in the large scale integration circuitry 83, the region 83a marked with the white dot expressed with the numeral “111” is provided with a conductor such as a TSV connected to the transmission and reception circuitry.

In the second modification example, as illustrated in the right section of FIG. 12, each of the regions (i.e., the four regions 81a, the four regions 82a, four regions 83a, and the four regions 84a) corresponding to all the transducer elements 20 is provided with the conductor such as the TSV connected to the corresponding transmission and reception circuitry. In other words, each of all the transducer elements 20 is connected to the corresponding transmission and reception circuitry. In another example, all the transducer elements 20 do not necessarily have to be connected to transmission and reception circuitry. For example, it is sufficient when the transmission and reception circuitries are connected to at least one of the transducer elements 20.

The ultrasound probe according to the second modification example has thus been explained. The ultrasound probe according to the second modification example is configured to ensure that, not one region, but the plurality of regions are provided as the area for each of the transmission and reception circuitries. For example, in the second modification example, it is ensured that the regions of which the quantity is equal to K are provided. As a result, according to the second modification example, it is possible to ensure, similarly to the first embodiment, that a sufficient circuitry area is provided for each of the transmission and reception circuitries in correspondence with a single transducer element 20.

### Second Embodiment

In the first embodiment, the example is explained in which the ultrasound probe 1 includes the plurality of large scale integration circuitry 22 and 23 provided with the transmission and reception circuitries 22b and 23b as the electronic circuitries having mutually the same functions. However, it is also acceptable to provide large scale integration circuitries with electronic circuitries having mutually-different functions. Thus, this embodiment will be explained as a second embodiment. Some of the constituent elements that are the same as those included in the ultrasound probe 1 according to the first embodiment will be referred to by using the same reference characters, and the explanations thereof may be omitted.

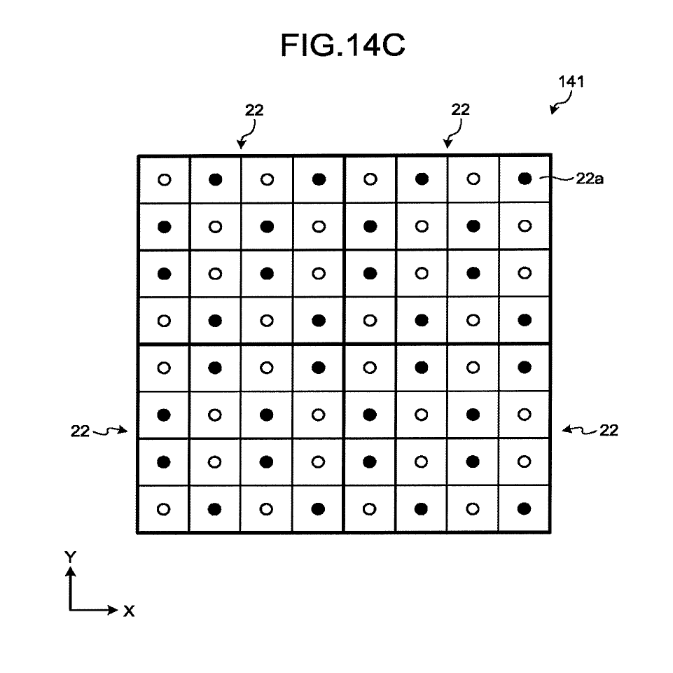

FIGS. 13A and 13B are drawings for explaining an example of an ultrasound probe according to the second embodiment. FIG. 13A illustrates large scale integration circuitry 130 according to the second embodiment. FIG. 13B illustrates large scale integration circuitry 131 according to the second embodiment. For example, the ultrasound probe according to the second embodiment is different from the ultrasound probe 1 according to the first embodiment for including the large scale integration circuitry 130 instead of the large scale integration circuitry 22 described above and for including the large scale integration circuitry 131 instead of the large scale integration circuitry 23 described above.

The controlling circuitry 31 according to the second embodiment has the same configuration as that of the controlling circuitry 31 according to the first embodiment. Thus, the explanations thereof will be omitted.

As illustrated in FIG. 13A, transmission circuitry 130b is provided in each of regions 130a of the large scale integration circuitry 130. In this situation, in the second embodiment, one transmission circuitry 130b is provided for each of the regions 130a. Each of the transmission circuitries 130b according to the second embodiment includes, for example, the pulser circuitry explained above and, from among the delay circuitries explained above, the circuitry (the transmission delay circuitry) configured to perform the delay process on the drive signal. In other words, each of the transmission circuitry 130b is circuitry related to the transmission of the ultrasound wave. The transmission circuitry 130b provided in the region 130a_1 will be referred to as “transmission circuitry 130b_1”. Further, the transmission circuitry 130b provided in the region 130a_2 will be referred to as “transmission circuitry 130b_2”.

Further, in FIG. 13A, the black dots depicted in the regions 130a schematically represent, for example, conductors each connecting the transmission circuitry 130b provided in the region 130a marked with the black dot to the controlling circuitry 31 positioned immediately below and to a region 131a positioned immediately above (explained later). In the second embodiment, a terminal and a TSV or the like are used as each of the conductors, for example. In that situation, in each of the regions 130a marked with the black dots, the transmission circuitry 130b is connected to the controlling circuitry 31 positioned immediately below, via the TSV or the like and is also connected to the region 131a positioned immediately above (explained later) (more specifically, to the transmission circuitry 130b positioned immediately above, explained later) via the terminal or the like.

As illustrated in FIG. 13B, reception circuitry 131b is provided in each of the regions 131a of the large scale integration circuitry 131. In this situation, in the second embodiment, one reception circuitry 131b is provided for each of the regions 131a. Each of the reception circuitries 131b according to the second embodiment includes, for example, the amplifier explained above and, from among the delay circuitries explained above, the circuitry (the reception delay circuitry) configured to perform the delay process on the echo signal. In other words, each of the transmission circuitry 130b is circuitry related to the reception of the ultrasound wave. The reception circuitry 131b provided in the region 131a_1 will be referred to as “reception circuitry 131b_1”. Further, the reception circuitry 131b provided in the region 131a_2 will be referred to as “reception circuitry 131b_2”.

Further, in FIG. 13B, the black dots depicted in the regions 131a schematically represent, for example, conductors each connecting the reception circuitry 131b provided in the region 131a marked with the black dot to the region 130a positioned immediately below (more specifically, to the transmission circuitry 130b provided in the region 130a positioned immediately below) and to the transducer element 20b positioned immediately above. In the second embodiment, a terminal and a TSV or the like are used as each of the conductors. In that situation, in each of the regions 131a marked with the black dots, the reception circuitry 131b is connected to the transmission circuitry 130b positioned immediately below via the TSV or the like and is also connected to the transducer element 20 positioned immediately above via the terminal or the like.

For example, in FIGS. 13A and 13B, under the control of the abovementioned controlling circuitry 31a, the transmission circuitry 130b_1 generates a drive signal and outputs the generated drive signal to the reception circuitry 131b positioned immediately above. After that, the reception circuitry 131b outputs the received drive signal to the transducer element 20_1 positioned immediately above.

Subsequently, the reception circuitry 131b_1 receives an echo signal output from the transducer element 20_1.

After that, under the control of the controlling circuitry 31a, the reception circuitry 131b_1 amplifies the received echo signal, performs a delay process on the amplified echo signal, and outputs the echo signal on which the delay process was performed to the transmission circuitry 130b_1. After that, the transmission circuitry 130b_1 outputs the received echo signal to the controlling circuitry 31a positioned immediately below.

The transmission circuitry 130b_2, the reception circuitry 131b_2, and the controlling circuitry 31b each also perform the same process. Further, the other transmission circuitries 130b, the other reception circuitries 131b, and the other controlling circuitries 31 each also perform the same process.

In this situation, the transmission circuitry 130b and the reception circuitry 131b have mutually-different withstand voltage levels. For example, in the transmission circuitry 130b, analogue circuitry such as pulse circuitry configured to generate the drive signal is dominant. In contrast, in the reception circuitry 131b, digital circuitry configured to process the echo signal generated by the transducer element 20 is dominant. For this reason, for example, the withstand voltage level of the transmission circuitry 130b is higher than the withstand voltage level of the reception circuitry 131b. In this situation, the reception circuitry 131b is an example of the first electronic circuitry, whereas the transmission circuitry 130b is an example of the second electronic circuitry.

For example, the types of manufacturing processes that can be used for manufacturing one large scale integration circuitry including a plurality of electronic circuitries having a large difference in the withstand voltage level thereof is limited to types of manufacturing processes with which it is possible to manufacture the electronic circuitry having the higher withstand voltage.

In this situation, the ultrasound probe according to the second embodiment is divided into the large scale integration circuitry 130 including the transmission circuitry 130b having a relatively high withstand voltage level and the large scale integration circuitry 131 including the reception circuitry 131b having a relatively low withstand voltage level. For this reason, when the large scale integration circuitry 131 is to be manufactured, the types of manufacturing processes are not limited by the level of the withstand voltage of the reception circuitries 131b included in the large scale integration circuitry 131. Accordingly, with the ultrasound probe according to the second embodiment, it is possible to avoid the situation where the types of manufacturing processes are limited.

Further, in the second embodiment, the various types of circuitries included in the transmission and reception circuitries 22b and 23b described above are divided into the transmission circuitries 130b and the reception circuitries 131b. Further, each of the electronic circuitries, namely the transmission circuitries 130b and the reception circuitries 131b, is provided in the single region 130a or 131a. As a result, according to the second embodiment, it is possible to further ensure that a sufficient circuitry area is provided for each of the transmission circuitries 130b and the reception circuitries 131b, compared to the situation where each of the transmission and reception circuitries 22b and 23b is provided in the single region 22a or 22b.

### Third Embodiment

In the first embodiment, the example is explained in which the ultrasound probe 1 includes the single large scale integration circuitry 22 and the single large scale integration circuitry 23. However, when a larger number of transducer elements 20 are used or the like, the ultrasound probe may include two or more of the large scale integration circuitries 22 and two or more of the large scale integration circuitries 23, in accordance with the quantity of the plurality of transducer elements 20. Thus, this example will be explained as a third embodiment.

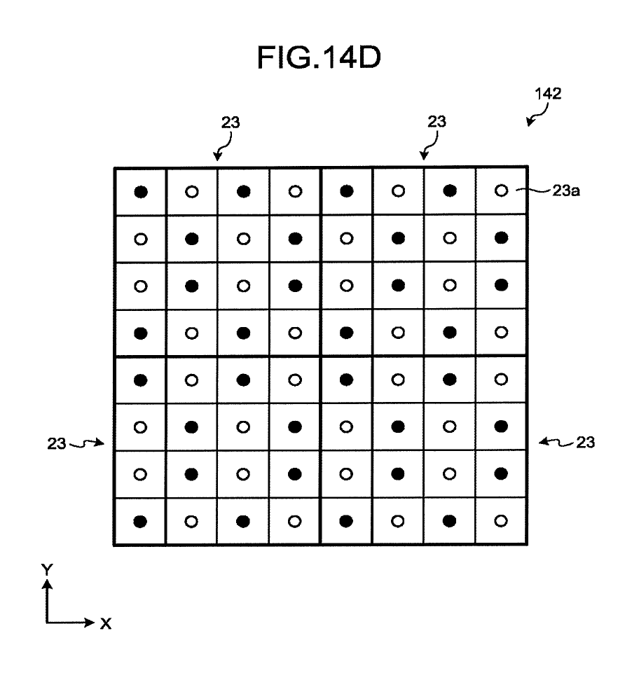

FIGS. 14A and 14B are drawing for explaining an example of an ultrasound probe according to the third embodiment. The ultrasound probe according to the third embodiment includes large scale integration circuitry 141 illustrated in FIG. 14A and large scale integration circuitry 142 illustrated in FIG. 14B.

In the large scale integration circuitry 141, three of the large scale integration circuitries 22 explained above are arranged in the X-axis direction in accordance with the quantity of the transducer elements 20. Further, in the large scale integration circuitry 142, three of the large scale integration circuitries 23 explained above are arranged in the X-axis direction, in accordance with the quantity of the transducer elements 20.

In the third embodiment, large scale integration circuitry provided with controlling circuitry configured to control the transmission and reception circuitries 22b provided for the large scale integration circuitry 141 and the transmission and reception circuitries 23b provided for the large scale integration circuitry 142 is newly manufactured in accordance with the quantity of the transducer elements 20.

A part of a method for manufacturing the ultrasound probe according to the third embodiment illustrated in FIGS. 14A and 14B will be explained. The plurality of (three in the example in FIG. 14B) large scale integration circuitries 142 are arranged in a predetermined orientation on the rear face side of the plurality of transducer elements 20. Further, the plurality of (three in the example in FIG. 14A) large scale integration circuitries 141 are arranged in an orientation different from the predetermined orientation on the rear face side of the large scale integration circuitries 142.

FIGS. 14C and 14D are drawing for explaining an another example of an ultrasound probe according to the third embodiment. As illustrated in FIG. 14C, in accordance with the quantity of the transducer elements 20, the large scale integration circuitry 141 may be structured so that, while each row includes two large scale integration circuitries 22 described above arranged in the X-axis direction, two such rows of large scale integration circuitries 22 are arranged in the Y-axis direction. Further, as illustrated in FIG. 14D, in accordance with the quantity of the transducer elements 20, the large scale integration circuitry 142 may be structured so that, while each row includes two large scale integration circuitries 23 described above arranged in the X-axis direction, two such rows of large scale integration circuitries 23 are arranged in the Y-axis direction.

In this situation, for example, when the quantity of the transducer elements 20 is 36 (i.e., while each row includes six transducer elements 20 arranged in the X-axis direction, six such rows are arranged in the Y-axis direction), it is acceptable to arrange, among the 64 regions 23a of the large scale integration circuitry 142, each of the 36 regions 23a in a central part to be connected to a different one of the 36 transducer elements 20. Further, it is acceptable to arrange, among the 64 regions 22a of the large scale integration circuitry 141, each of the 36 regions 22a in a central part to be connected to a different one of the 36 regions 23a described above. Further, it is acceptable to arrange each of the 36 regions 22a in the central part to be connected to a different one of the 36 controlling circuitries 31.

### Fourth Embodiment

In the first embodiment, the example is explained in which, after the large scale integration circuitry 23 is rotated with respect to the large scale integration circuitry 22, the large scale integration circuitry 23 is arranged in the predetermined orientation, so that the large scale integration circuitry 22 is arranged in the orientation different from the predetermined orientation.

However, another arrangement is also acceptable in which, with respect to one of the large scale integration circuitries, after the other large scale integration circuitry is moved (shifted) in at least one selected from between the X-axis direction and the Y-axis direction, the one large scale integration circuitry and the other large scale integration circuitry that has been moved are arranged.

By using the ultrasound probe and the ultrasound probe manufacturing method according to at least one aspect of the embodiments described above, it is possible to ensure that sufficient circuitry areas are provided for the electronic circuitries.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. An ultrasound probe comprising:
a plurality of transducer elements each configured to perform at least one selected from between transmission of an ultrasound wave and reception of an ultrasound wave;
first circuitry that is arranged on a rear face side of the plurality of transducer elements, is provided with an electronic circuitry corresponding to two or more of the plurality of transducer elements organized in a first group, and is provided with a through silicon via corresponding to two or more of the plurality of transducer elements organized in a second group; and
second circuitry that is arranged on a rear face side of the first circuitry, is provided with a through silicon via corresponding to the two or more of the transducer elements organized in the first group, and is provided with an electronic circuitry corresponding to the two or more of the transducer elements organized in the second group.

2. The ultrasound probe according to claim 1, wherein the electronic circuitries provided for the first and the second circuitries include analog circuitry.

3. The ultrasound probe according to claim 1, wherein the electronic circuitries provided for the first and the second circuitries include at least one selected from between: a driving circuitry configured to drive at least one of the transducer elements; and signal processing circuitry configured to process an electrical signal generated by said at least one of the transducer elements.

4. The ultrasound probe according to claim 1, wherein the first circuitry and the second circuitry are identical circuitries that are arranged in mutually-different orientations.

5. The ultrasound probe according to claim 1, wherein the first circuitry and the second circuitry are each provided with a marker indicating an orientation thereof.

6. The ultrasound probe according to claim 1, wherein
a plurality of through silicon vias provided for the first circuitry are connected to a plurality of electronic circuitries provided for the second circuitry, and
a plurality of electronic circuitries provided for the first circuitry are connected to a plurality of through silicon via provided for the second circuitry.

7. The ultrasound probe according to claim 1, comprising: controlling circuitry that is arranged on a rear face side of the second circuitry and is configured to control the electronic circuitries provided for the first and the second circuitries.

8. An ultrasound probe comprising:
a plurality of transducer elements each configured to perform at least one selected from between transmission of an ultrasound wave and reception of an ultrasound wave;
first circuitry that is arranged on a rear face side of the plurality of transducer elements and is provided with first electronic circuitry corresponding to each of the plurality of transducer elements; and
second circuitry that is arranged on a rear face side of the first circuitry and is provided with second electronic circuitry corresponding to each of the plurality of transducer elements, wherein
the first electronic circuitry and the second electronic circuitry have mutually-different withstand voltage levels.

9. An ultrasound probe comprising:
a plurality of transducer elements each configured to perform at least one selected from between transmission of an ultrasound wave and reception of an ultrasound wave;
first circuitry that is arranged on a rear face side of the plurality of transducer elements and is provided with first electronic circuitry corresponding to each of the plurality of transducer elements; and
second circuitry that is arranged on a rear face side of the first circuitry and is provided with second electronic circuitry corresponding to each of the plurality of transducer elements, wherein
one selected from between the first electronic circuitry and the second electronic circuitry is circuitry related to the transmission of the ultrasound wave, and
the other of the first and the second electronic circuitries is circuitry related to the reception of the ultrasound wave.

10. An ultrasound probe comprising:
a plurality of transducer elements each configured to perform at least one selected from between transmission of an ultrasound wave and reception of an ultrasound wave;
first circuitry that is arranged on a rear face side of the plurality of transducer elements and is provided with electronic circuitry connected to at least one of the plurality of transducer elements; and
second circuitry that is arranged on a rear face side of the first circuitry and is provided with electronic circuitry connected to at least one of the plurality of transducer elements, wherein
the first circuitry and the second circuitry are identical circuitries that are arranged in mutually-different orientations.

11. The ultrasound probe according to claim 1, further comprising a relay substrate that is provided between the plurality of transducer elements and the first circuitry and is configured to relay connections between the plurality of transducer elements and the electronic circuitry provided for the first circuitry.

12. An ultrasound probe manufacturing method for manufacturing an ultrasound probe including a plurality of transducer elements each configured to perform at least one selected from between transmission of an ultrasound wave and reception of an ultrasound wave, first circuitry provided with electronic circuitry connected to at least one of the plurality of transducer elements, and second circuitry provided with electronic circuitry connected to at least one of the plurality of transducer elements, the ultrasound probe manufacturing method comprising:
arranging the first circuitry on a rear face side of the plurality of transducer elements to be in a first orientation; and
arranging the second circuitry on a rear face side of the first circuitry to be in a second orientation different from the first orientation, wherein
the first circuitry and the second circuitry are identical circuitries.

13. The ultrasound probe manufacturing method according to claim 12, wherein
two or more of the first circuitries are arranged to be in the first orientation, and
two or more of the second circuitries are arranged to be in the second orientation.
